(19) United States
(12) Patent Application Publication
(10) Pub. No.: US 2009/0268953 A1
(43) Pub. Date: Oct. 29, 2009

(54) Method for the automatic adjustment of image parameter settings in an imaging system

(75) Inventor: Kevin M. CRUCS, Copley, OH (US)

Correspondence Address: HAHN LOESER & PARKS, LLP, One GOJO Plaza, Suite 300, AKRON, OH, 44311-1076, US

(73) Assignee: APTERYX, INC., Akron, OH (US)

(21) Appl. No.: 12/108,736

(22) Filed: Apr. 24, 2008

(51) Int. Cl. A61B 5/00; G06K 9/03

(52) U.S. Cl. 382/128; 382/255

(57) ABSTRACT

A system, method, and computer readable medium for facilitating the automatic adjustment of image parameter settings in an imaging system having a sensor subsystem. An imaging mode of operation of the imaging system is selected and entered. A current set of digital image data of an imaging phantom device is acquired with the imaging system via the sensor subsystem and processed to generate a current set of image-processed data using the imaging system. The current set of image-processed data is automatically compared to a previous set of image-processed data or a target set of specifications, representing a standard of image quality and corresponding to the selected imaging mode of operation. At least one image parameter setting may be automatically adjusted to account for a difference in at least one image parameter between the current set of image-processed data and the previous set of image processed data or target set of specifications.

### TECHNICAL FIELD

Certain embodiments relate to image quality. More particularly, certain embodiments relate to automatically adjusting image parameter settings in an imaging system having a sensor subsystem by imaging a phantom device.

### BACKGROUND

Various types of imaging systems are available for imaging the surface and/or the interior of such diverse entities such as, for example, the human anatomy, animals, man-made physical structures such as welding joints in bridges, geological formations, bodies of water, as well as many others. For example, in the field of medical imaging, various types of sensors exist which are used for acquiring image data of various anatomical portions of the human body.

The image quality produced by an imaging system may change or degrade over time as various image parameter settings of the imaging system are changed by operators or technicians, or as a sensor subsystem of the imaging system degrades over time. For example, a noise filter setting may be inadvertently or deliberately changed by an operator, resulting in a noisier image. A contrast setting may be inadvertently or deliberately changed by an operator, resulting in less contrast resolution. Image quality degradation may not be noticed immediately by an operator, especially if the degradation occurs gradually over time. Furthermore, once the image quality degradation is noticed, it can be time consuming and difficult to track down the source of the image quality degradation in order to bring image quality back up to a desired level.

Further limitations and disadvantages of conventional, traditional, and proposed approaches will become apparent to one of skill in the art, through comparison of such approaches with the subject matter of the present application as set forth in the remainder of the present application with reference to the drawings.

### BRIEF SUMMARY

A first embodiment comprises a method for the automatic adjustment of image parameter settings in an imaging system having a sensor subsystem, providing at least one imaging mode of operation, and storing at least one previous set of image-processed data corresponding to at least one previously acquired set of digital image data of an imaging phantom device and representing a standard image of quality corresponding to the at least one imaging mode of operation, the method comprising:
(a) selecting and entering an imaging mode of operation of the imaging system;
(b) positioning an imaging phantom device with respect to the sensor subsystem;
(c) acquiring a current set of digital image data of the imaging phantom device with the imaging system via the sensor subsystem;
(d) processing the current set of acquired digital image data to generate a current set of image-processed data using the imaging system;
(e) automatically comparing the current set of image-processed data to the previous set of image-processed data representing a standard image of quality corresponding to the selected imaging mode of operation using the imaging system;
(f) automatically determining, in response to the comparing, at least one imaging parameter difference using the imaging system;
(g) automatically adjusting at least one image parameter setting of the imaging system in response to the at least one imaging parameter difference and generating an updated current set of image-processed data based on the at least one adjusted image parameter setting using the imaging system if said at least one imaging parameter difference is not minimized; and
(h) automatically repeating steps (e) through (g) until the at least one imaging parameter difference is minimized.

The at least one image parameter setting may include one of a noise filter setting, a brightness contrast setting, a gamma setting, a brightness leveling setting, and a contrast leveling setting. Color settings such as, for example, a HSL (hue saturation lightness) setting, a HSV (hue-saturation-value) setting, a HSI (hue-saturation-intensity) setting, a HSB (hue-saturation-brightness) setting, an RGB (red-green-blue) setting, and a CMYK (Cyan-Magenta-Yellow-Key/blacK) setting may also be included in an imaging system. Other image parameter settings are possible as well.

Another embodiment comprises a computer readable medium having encoded thereon computer executable instructions for performing a method for the automatic adjustment of image parameter settings in an imaging system having a sensor subsystem, providing at least one imaging mode of operation, and storing at least one previous set of image-processed data corresponding to at least one previously acquired set of digital image data of an imaging phantom device and representing a standard image of quality corresponding to the at least one imaging mode of operation, wherein the method comprises:
(a) selecting and entering an imaging mode of operation of the imaging system;
(b) acquiring a current set of digital image data of the imaging phantom device with the imaging system via the sensor subsystem;
(c) processing the current set of acquired digital image data to generate a current set of image-processed data using the imaging system;
(d) automatically comparing the current set of image-processed data to the previous set of image-processed data representing a standard image of quality corresponding to the selected imaging mode of operation using the imaging system;
(e) automatically determining, in response to the comparing, at least one imaging parameter difference using the imaging system;
(f) automatically adjusting at least one image parameter setting of the imaging system in response to the at least one imaging parameter difference and generating an updated current set of image-processed data based on the at least one adjusted image parameter setting using the imaging system if said at least one imaging parameter difference is not minimized; and
(g) automatically repeating steps (d) through (f) until the at least one imaging parameter difference is minimized.

The computer readable medium may include, for example, a digital memory, a compact disk (CD), a memory stick, a magnetic tape, or any other computer readable medium.

The at least one image parameter setting may include one of a noise filter setting, a brightness contrast setting, a gamma setting, a brightness leveling setting, and a contrast leveling setting. Color settings such as, for example, a HSL (hue saturation lightness) setting, a HSV (hue-saturation-value) setting, a HSI (hue-saturation-intensity) setting, a HSB (hue-saturation-brightness) setting, an RGB (red-green-blue) setting, and a CMYK (Cyan-Magenta-Yellow-Key/blacK) setting may also be included in an imaging system. Other image parameter settings are possible as well.

A further embodiment comprises an imaging system having a sensor subsystem, an image processor, and a controller, providing at least one imaging mode of operation, and storing at least one previous set of image-processed data corresponding to at least one previously acquired set of digital image data of an imaging phantom device and representing a standard image of quality corresponding to the at least one imaging mode of operation, and having encoded therein computer executable instructions for performing a method for the automatic adjustment of image parameter settings, wherein the method comprises:
(a) selecting and entering an imaging mode of operation of the imaging system;
(b) acquiring a current set of digital image data of an imaging phantom device with the imaging system via the sensor subsystem;
(c) processing the current set of acquired digital image data to generate a current set of image-processed data;
(d) automatically comparing the current set of image-processed data to the previous set of image-processed data representing a standard image of quality corresponding to the selected imaging mode of operation;
(e) automatically determining, in response to the comparing, at least one imaging parameter difference;
(f) automatically adjusting at least one image parameter setting of the imaging system in response to the at least one imaging parameter difference and generating an updated current set of image-processed data based on the at least one adjusted image parameter setting if said at least one imaging parameter difference is not minimized; and
(g) automatically repeating steps (d) through (f) until the at least one imaging parameter difference is minimized.

The sensor subsystem may include an X-ray tube with associated transmitting circuitry and an X-ray detector with associated receiving circuitry. The at least one image parameter setting may include one of a noise filter setting, a brightness contrast setting, a gamma setting, a brightness leveling setting, and a contrast leveling setting. Color settings such as, for example, a HSL (hue saturation lightness) setting, a HSV (hue-saturation-value) setting, a HSI (hue-saturation-intensity) setting, a HSB (hue-saturation-brightness) setting, an RGB (red-green-blue) setting, and a CMYK (Cyan-Magenta-Yellow-Key/blacK) setting may also be included in an imaging system. Other image parameter settings are possible as well.

Another embodiment comprises a method for the automatic adjustment of image parameter settings in an imaging system having a sensor subsystem, providing at least one imaging mode of operation, and storing at least one target specification representing a standard of image quality and corresponding to the at least one imaging mode of operation, the method including:
(a) selecting and entering an imaging mode of operation of the imaging system;
(b) positioning an imaging phantom device with respect to the sensor subsystem;
(c) acquiring a current set of digital image data of the imaging phantom device with the imaging system via the sensor subsystem;
(d) processing the current set of acquired digital image data to generate a current set of image-processed data using the imaging system;
(e) automatically comparing the current set of image-processed data to the at least one target specification representing a standard of image quality and corresponding to the selected imaging mode of operation using the imaging system;
(f) automatically determining, in response to the comparing, at least one imaging parameter difference using the imaging system;
(g) automatically adjusting at least one image parameter setting of the imaging system in response to the at least one imaging parameter difference and generating an updated current set of image-processed data based on the at least one adjusted image parameter setting using the imaging system if the at least one imaging parameter difference is not minimized; and
(h) automatically repeating steps (e) through (g) until the at least one imaging parameter difference is minimized.

A further embodiment comprises a computer readable medium having encoded thereon computer executable instructions for performing a method for the automatic adjustment of image parameter settings in an imaging system having a sensor subsystem, providing at least one imaging mode of operation, and storing at least one target specification representing a standard of image quality corresponding to the at least one imaging mode of operation, wherein the method comprises:
(a) selecting and entering an imaging mode of operation of the imaging system;
(b) acquiring a current set of digital image data of the imaging phantom device with the imaging system via the sensor subsystem;
(c) processing the current set of acquired digital image data to generate a current set of image-processed data using the imaging system;
(d) automatically comparing the current set of image-processed data to the at least one target specification representing a standard of image quality corresponding to the selected imaging mode of operation using the imaging system;
(e) automatically determining, in response to the comparing, at least one imaging parameter difference using the imaging system;
(f) automatically adjusting at least one image parameter setting of the imaging system in response to the at least one imaging parameter difference and generating an updated current set of image-processed data based on the at least one adjusted image parameter setting using the imaging system if the at least one imaging parameter difference is not minimized; and
(g) automatically repeating steps (d) through (f) until the at least one imaging parameter difference is minimized.

Another embodiment comprises an imaging system having a sensor subsystem, an image processor, and a controller, providing at least one imaging mode of operation, and storing at least one target specification representing a standard of image quality corresponding to the at least one imaging mode of operation, and having encoded therein computer executable instructions for performing a method for the automatic adjustment of image parameter settings, wherein the method comprises:
(a) selecting and entering an imaging mode of operation of the imaging system;
(b) acquiring a current set of digital image data of an imaging phantom device with the imaging system via the sensor subsystem;
(c) processing the current set of acquired digital image data to generate a current set of image-processed data;
(d) automatically comparing the current set of image-processed data to the at least one target specification representing a standard of image quality corresponding to the selected imaging mode of operation;
(e) automatically determining, in response to the comparing, at least one imaging parameter difference;
(f) automatically adjusting at least one image parameter setting of the imaging system in response to the at least one imaging parameter difference and generating an updated current set of image-processed data based on the at least one adjusted image parameter setting if the at least one imaging parameter difference is not minimized; and
(g) automatically repeating steps (d) through (f) until the at least one imaging parameter difference is minimized.

These and other novel features of the subject matter of the present application, as well as details of illustrated embodiments thereof, will be more fully understood from the following description and drawings.

### BRIEF DESCRIPTION OF THE DRAWINGS

FIGS. 1A-1B illustrate a schematic diagram of an exemplary embodiment of an imaging phantom device;

FIG. 2 illustrates a table listing a plurality of exemplary imaging parameters and image parameter settings;

FIG. 3 illustrates a schematic diagram of a first exemplary embodiment of an imaging system having a sensor subsystem and an image processor and controller, providing at least one imaging mode of operation, and at least one previous set of image-processed data corresponding to at least one previously acquired set of digital image data of the imaging phantom device of FIG. 1 stored in memory and representing a standard image of quality;

FIG. 4 illustrates a schematic diagram of a second exemplary embodiment of an imaging system having a sensor subsystem and an image processor and controller, providing at least one imaging mode of operation, and at least one previous set of image-processed data corresponding to at least one previously acquired set of digital image data of the imaging phantom device of FIG. 1 stored in memory and representing a standard image of quality;

FIG. 5 illustrates a flowchart of a first exemplary embodiment of a method for the automatic adjustment of image parameter settings in the imaging system of FIG. 3 or FIG. 4 using the imaging phantom device of FIG. 1; and

FIG. 6 illustrates a flowchart of a second exemplary embodiment of a method for the automatic adjustment of image parameter settings in the imaging system of FIG. 3 or FIG. 4 using the imaging phantom device of FIG. 1.

### DETAILED DESCRIPTION

The following description is presented in the context of medical X-ray imaging and medical ultrasound imaging. However, various embodiments may be applied to other imaging fields as well such as, for example, other branches of medical imaging including magnetic resonance imaging, positron emission tomography, various forms of computed tomography, and others.

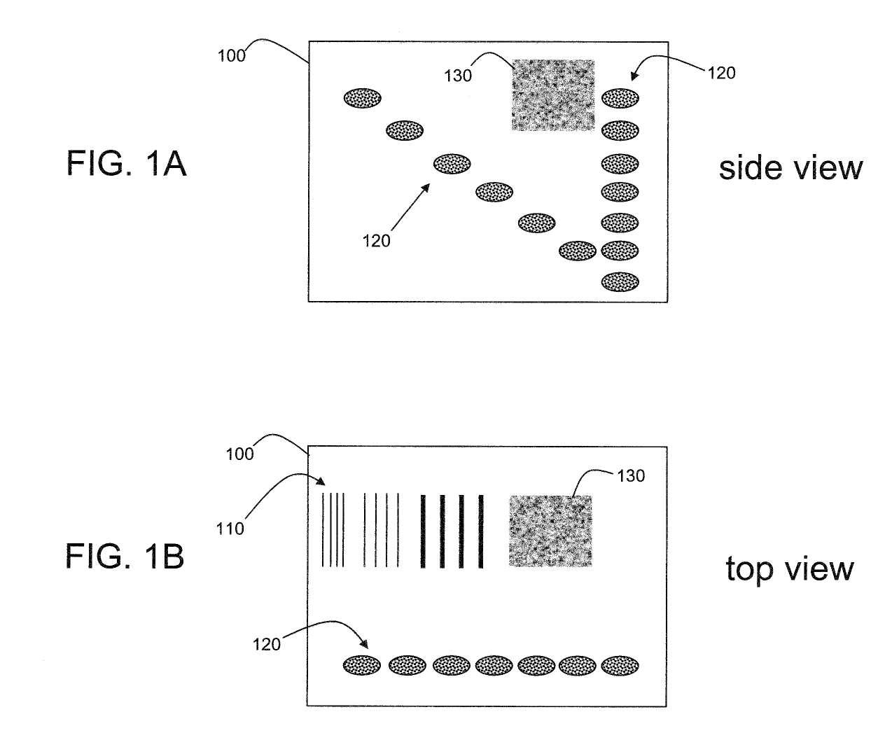

FIGS. 1A-1B illustrate a schematic diagram of an exemplary embodiment of an imaging phantom device 100. FIG. 1A shows a side view of the imaging phantom device 100 and FIG. 1B shows a top view of the imaging phantom device 100. The imaging phantom device 100 includes various internal features 110-130. The imaging phantom device 100 shown herein is for illustrative purposes and discussion purposes only and is not meant to correspond to any particular imaging phantom device or any particular imaging modality.

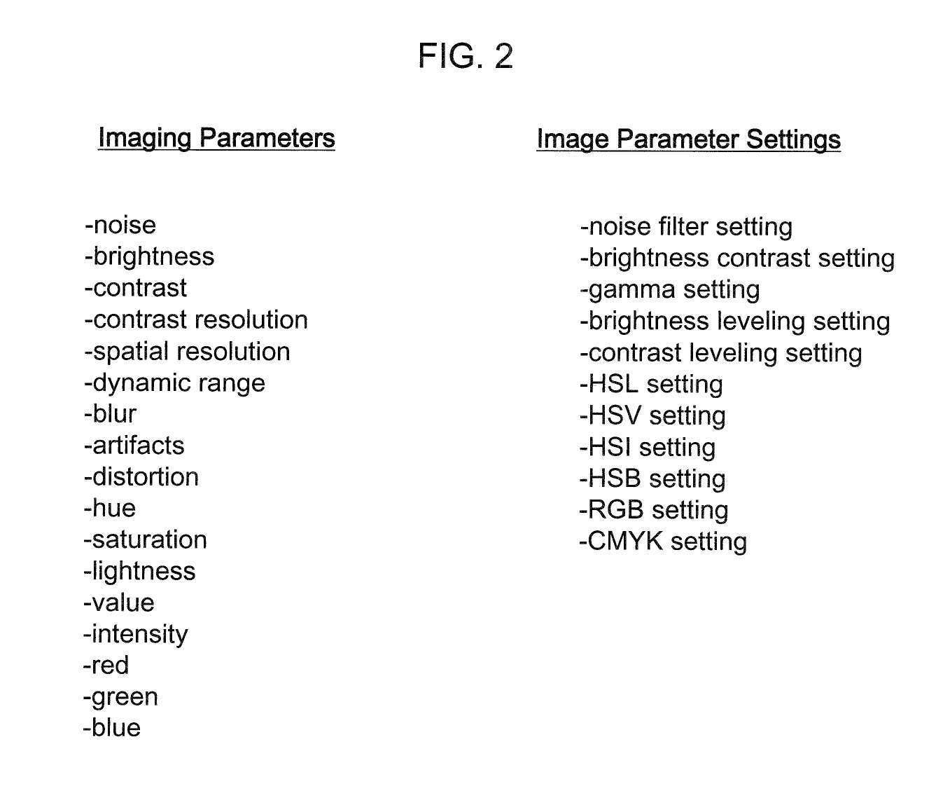

The imaging phantom device 100 may be designed to include various features that, when imaged, allow various imaging parameters to be determined which correlate to various image parameter settings. FIG. 2 illustrates a table listing a plurality of exemplary imaging parameters and image parameter settings. Such imaging parameters may include noise, brightness, contrast, and contrast resolution. Other imaging parameters are possible as well such as, for example, spatial resolution, dynamic range, blur, artifacts, and distortion. Further imaging parameters may include hue, saturation, lightness, value, intensity, red, green, and blue. Such imaging parameters are well-known in the art.

Such image parameter settings may include, for example, a noise filter setting of an imaging system, a brightness contrast setting of an imaging system, a gamma setting of an imaging system, a brightness leveling setting of an imaging system, and a contrast leveling setting of an imaging system. Other image parameter settings are possible as well such as, for example, color settings including a HSL (hue-saturation-lightness) setting of an imaging system, a HSV (hue-saturation-value) setting of an imaging system, a HIS (hue-saturation-intensity) setting of an imaging system, a HSB (hue-saturation-brightness) setting of an imaging system, a RGB (red-green-blue) setting of an imaging system, and a CMYK (Cyan-Magenta-Yellow-Key/blacK) setting of an imaging system. Such image parameter settings are well-known in the art.

For example, for an X-ray imaging system, the sensor subsystem may include an X-ray tube and an X-ray detector, along with the corresponding transmitting circuitry and receiving circuitry. The X-ray system may further include an image processor and controller and a display device. In such an X-ray system, an image parameter setting may include a noise filter setting within the image processor and controller or display device. The image processor and controller or display device may include a plurality of selectable noise filter settings. Such noise filter settings may each select a digital noise filter designed to filter out a particular type of image noise such as, for example, quantum noise or electric noise.

Another image parameter setting may include a brightness contrast setting within the image processor and controller or display device. Brightness contrast, also known as lightness contrast, is the apparent darkening of an object or image when viewed against, alongside, or immediately after a lighter object or image, or an apparent lightening of an object or image juxtaposed with a darker object or image. The image processor and controller or display device may include a plurality of selectable brightness contrast settings each designed to provide a different amount of brightness contrast.

A further image parameter setting may include a gamma setting within the image processor and controller or display device. Gamma defines a transfer function between an input pixel brightness and an output or displayed pixel brightness of an image. The image processor and controller or display device may include a plurality of selectable gamma settings each designed to provide a different transfer function between input pixel brightness and output pixel brightness. For example, a selected gamma setting may correct for a transfer function of the display device that inherently provides an undesirable relationship between input pixel brightness and output pixel brightness.

Another image parameter setting may include a brightness leveling setting within the image processor and controller or display device. Brightness leveling, as used herein, involves applying a subset of a range of gray scale or color values to the image data to improve the overall brightness of an image. For example, instead of applying a full gray scale range of 0 to 255 (where 0 represents black and 255 represents white) to the pixels of an image, a subset or sub-range of 50 to 200 shades of gray may be applied. The image processor and controller or display device may include a plurality of selectable brightness leveling settings each designed to provide a different sub-range of brightness levels (i.e., gray scale or color levels).

A further image parameter setting may include a contrast leveling setting within the image processor and controller or display device. Contrast leveling, as used herein, involves applying a full range of gray scale or color values to a subset of the image data to improve image contrast. For example, instead of applying the full gray scale range of 0 to 255 (where 0 represents black and 255 represents white) to a full image data range of 0 to 2000, the full gray scale range may be applied to a subset or sub-range of 1000 to 1080 of the image data, for example. The image processor and controller or display device may include a plurality of selectable contrast leveling settings each designed to provide a different sub-range of image data. Contrast leveling allows features of interest to be emphasized in an image.

Another image parameter setting may include a RGB setting within the image processor and controller or display device. An RGB setting may correspond to, for example, a particular color map to be applied to the acquired image data. The image processor and controller or display device may include a plurality of selectable RGB settings each designed to provide a different color map to be applied to the image data.

Other image parameter settings may include HSL or HSV settings within the image processor and controller or display device. HSL and HSV are two related representations of points in an RGB color space that attempt to describe perceptual color relationships more accurately than RGB, while remaining computationally simple. HSI and HSB are alternative names for such concepts, using intensity and brightness. Furthermore, another image parameter setting may be a CMYK setting within the image processor and controller or display device. Such settings may correspond to, for example, a particular color representation to be applied to the acquired image data. The image processor and controller or display device may include a plurality of selectable settings (e.g., HSL, HSV, HSI, HSB, or CMYK) each designed to provide a different color representation to be applied to the image data.

Referring to FIGS. 1A-1B, the feature 110 of the imaging phantom device 100 may include a set of reflective line pairs which allow determination of imaging system brightness, spatial resolution, blur, and distortion when imaged. Brightness is related to a brightness contrast setting and a brightness leveling setting of the imaging system. Similarly, the feature 120 may include a set of energy absorbing volumes, staggered over a depth of the phantom device 100, which allow determination of imaging system penetration, sensitivity, and noise. Noise is related to a noise filter setting of the imaging system. Furthermore, the feature 130 may include a volume of varying density, reflectivity, and attenuation which allows determination of imaging system brightness, contrast, and contrast resolution. Contrast and contrast resolution are related to a gamma setting and a contrast leveling setting of the imaging system. Other features may be included in the imaging phantom device 100 as well for helping to determine various imaging parameters. Imaging phantom devices are well-known in the medical sensor imaging art as well as other sensor imaging arts as well.

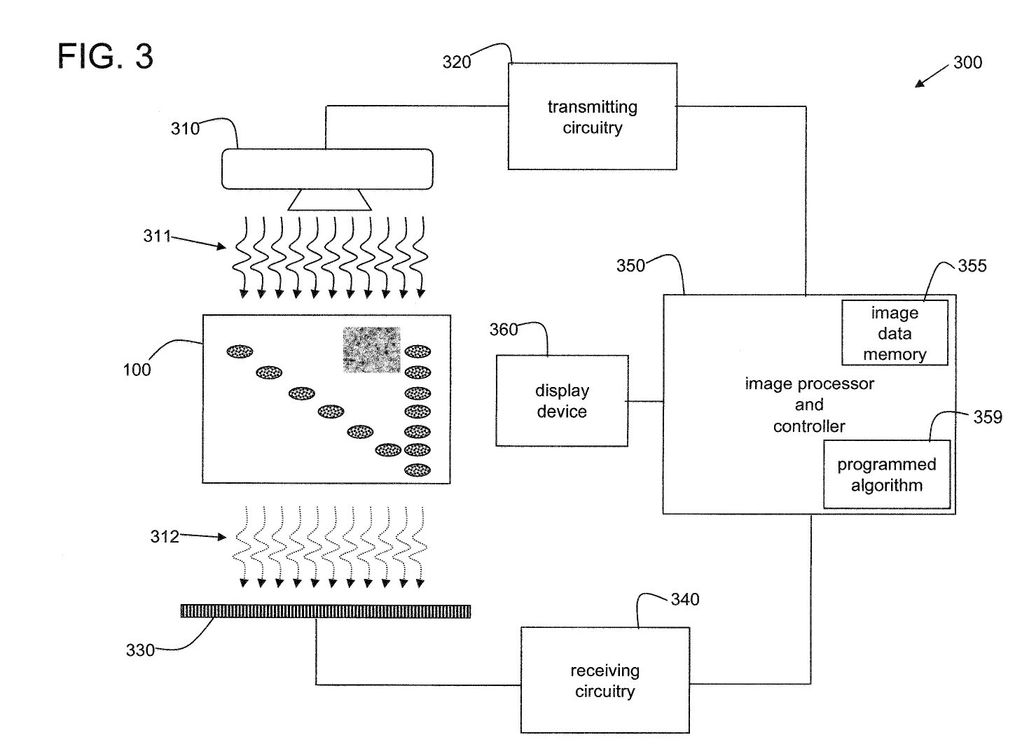

FIG. 3 illustrates a schematic block diagram of a first exemplary embodiment of an imaging system 300 having a sensor subsystem and an image processor and controller, providing at least one imaging mode of operation, and at least one previous set of image-processed data corresponding to at least one previously acquired set of digital image data of the imaging phantom device 100 of FIG. 1 stored in image data memory and being representative of a standard image of quality. The sensor subsystem of the imaging system 300 includes an X-ray tube 310, transmitting circuitry 320 operationally connected to the X-ray tube 310, an X-ray detector or sensor 330, and receiving circuitry 340 operationally connected to the X-ray detector 330. Such X-ray tubes, transmitting circuitry, X-ray detectors, and receiving circuitry are well known in the art.

The imaging system 300 further includes an image processor and controller 350 operationally interfacing to the transmitting circuitry 320 and the receiving circuitry 340. The image processor and controller 350 is capable of being programmed with computer software instructions for controlling the transmitting circuitry and the receiving circuitry, and for performing image processing and image parameter adjustment functions as described herein. The image processor and controller 350 includes an image data memory 355 for storing acquired digital image data and corresponding processed digital image data. Such image processor and controllers are well known in the art.

The image processor and controller 350 is also programmed with an algorithm 359 which is used to perform at least a portion of the methods for the automatic adjustment of image parameter settings in an imaging system as described herein and, therefore, makes the image processor and controller 350 a unique special purpose image processor and controller, in accordance with an embodiment of the present invention.

The imaging phantom device 100 may be positioned between the X-ray tube 310 and the X-ray detector 330. The X-ray tube 310 is capable of generating X-ray radiation 311 which penetrates through the phantom device 100 such that a resulting attenuated X-ray radiation 312 may be received at the X-ray detector 330. The imaging system 300 further includes a display device 360 for displaying processed images and for displaying messages and image quality test results to an operator.

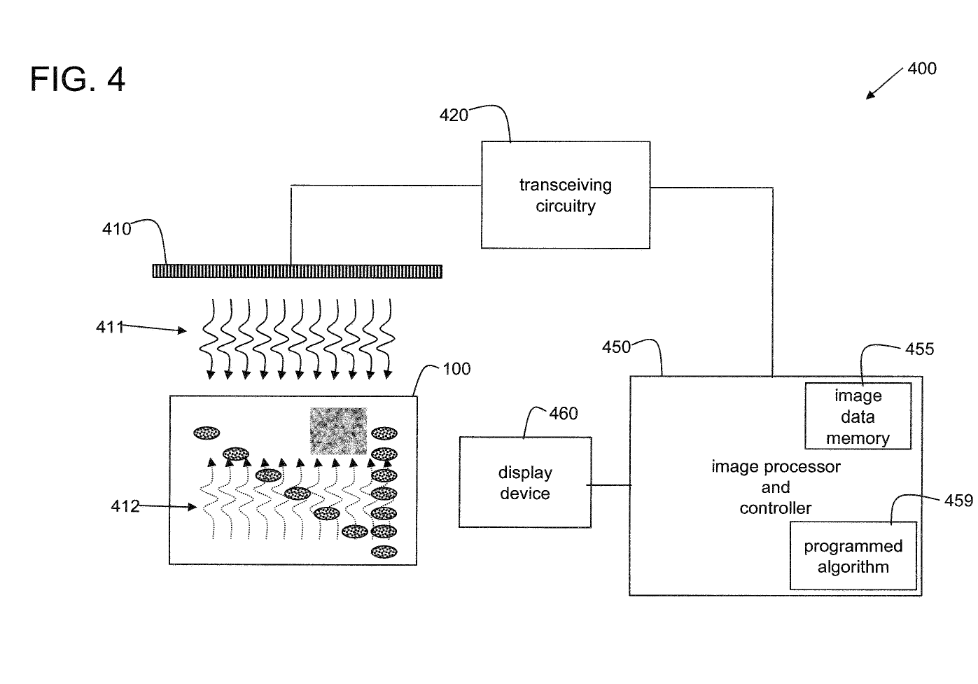

FIG. 4 illustrates a schematic diagram of a second exemplary embodiment of an imaging system 400 having a sensor subsystem and an image processor and controller, providing at least one imaging mode of operation, and at least one previous set of image-processed data corresponding to at least one previously acquired set of digital image data of the imaging phantom device of FIG. 1 stored in image data memory and being representative of a standard image of quality. The sensor subsystem of the imaging system 300 includes an ultrasound transducer 410 and transceiving circuitry 420 operationally connected to the ultrasound transducer 410. Such ultrasound transducers and transceiving circuitry are well known in the art.

The imaging system 400 further includes an image processor and controller 450 operationally interfacing to the transceiving circuitry 420. The image processor and controller 450 is capable of being programmed with computer software instructions for controlling the transceiving circuitry, and for performing image processing and image parameter adjustment functions as described herein. The image processor and controller 450 includes an image data memory 455 for storing acquired digital image data and corresponding processed digital image data. Such image processor and controllers are well known in the art.

The image processor and controller 450 is also programmed with an algorithm 459 which is used to perform at least a portion of the methods for the automatic adjustment of image parameter settings in an imaging system as described herein and, therefore, makes the image processor and controller 450 a unique special purpose image processor and controller, in accordance with an embodiment of the present invention.

The imaging phantom device 100 may be positioned with respect to the ultrasound transducer 410. The ultrasound transducer 410 is capable of generating ultrasound energy 411 which penetrates into the phantom device 100 such that a resulting reflected and attenuated ultrasound energy 412 may be received back at the ultrasound transducer 410 in a time delayed manner. Typically, the transducer 410 is placed in physical and acoustic contact with the phantom device 100 in order to couple the ultrasound energy into the phantom device 100. The imaging system 400 further includes a display device 460 for displaying processed images and for displaying messages and image quality test results to an operator.

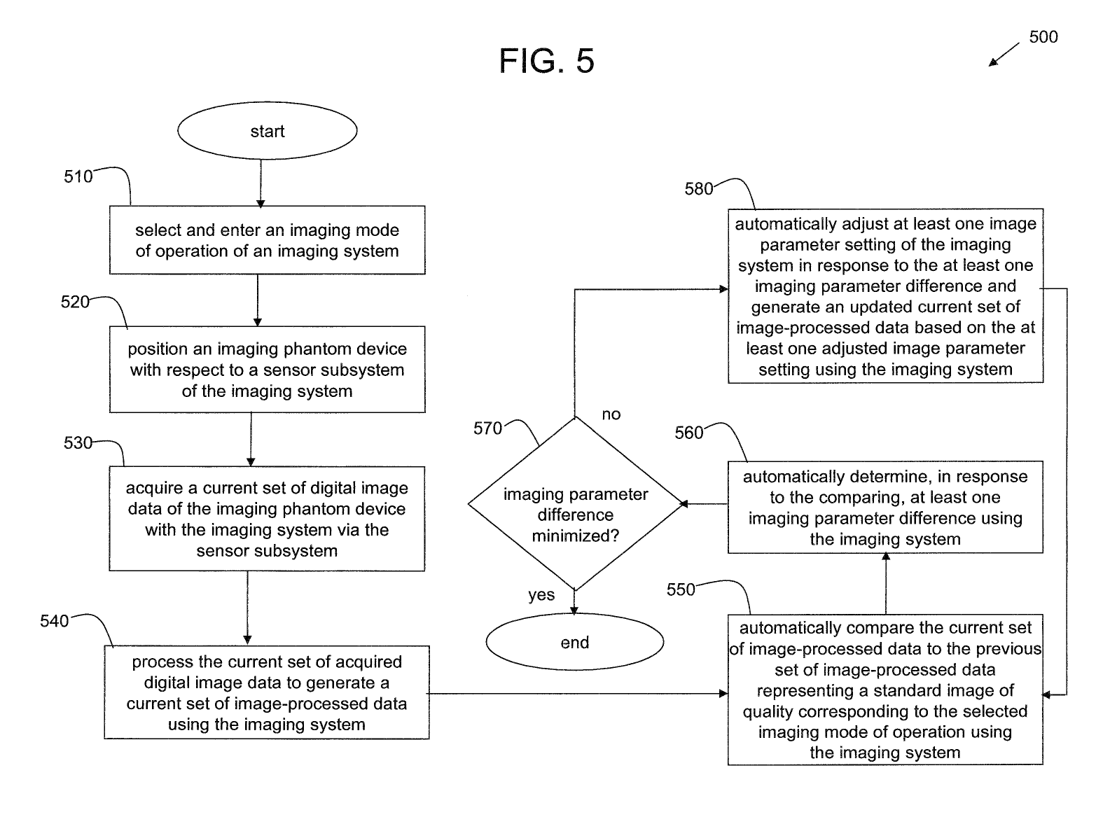

FIG. 5 illustrates a flowchart of a first exemplary embodiment of a method 500 for the automatic adjustment of image parameter settings in the imaging systems 300 or 400 of FIG. 3 or FIG. 4 using the imaging phantom device 100 of FIG. 1. The method 500 constitutes an image adjustment or image calibration routine (e.g., the algorithm 359) that may be initiated by an operator or technician of the imaging system. In step 510, select and enter an imaging mode of operation of an imaging system. For example, the imaging modality may be that of medical X-ray and the selected imaging mode of operation may be that of a chest X-ray. In step 520, position an imaging phantom device with respect to a sensor subsystem of the imaging system. In step 530, acquire a current set of digital image data of the imaging phantom device with the imaging system via the sensor subsystem. In step 540, process the current set of acquired digital image data to generate a current set of image-processed data using the imaging system. In step 550, automatically compare the current set of image-processed data to the previous set of image-processed data representing a standard image of quality corresponding to the selected imaging mode of operation using the imaging system.

In step 560, automatically determine, in response to the comparing, at least one imaging parameter difference using the imaging system. For example, the imaging parameter difference may correspond to a difference in contrast resolution between the current set of image-processed data and the previous set of image-processed data (i.e., the standard). In step 570, determine if the at least one imaging parameter difference is minimized (e.g., is below a predetermined minimum threshold value). If the imaging parameter difference is minimized, then end the method 500. If the imaging parameter difference is not minimized, then in step 580, automatically adjust at least one image parameter setting of the imaging system in response to the at least one imaging parameter difference and generate an updated current set of image-processed data based on the at least one adjusted image parameter setting using the imaging system. For example, if the imaging parameter difference corresponds to a difference in contrast resolution which is too large (i.e., not minimized), then a contrast leveling setting may be adjusted. Then go back and repeat steps 550 to 580 until the at least one imaging parameter difference is minimized.

The previous set of image-processed data represents a standard image of quality for the selected imaging mode of operation. The standard image of quality is derived from an acquired image of the phantom device 100 and represents the desired level of image quality (i.e., the established standard) for the selected imaging mode of operation. The previously acquired set of digital phantom image data may have been previously acquired using the exact same or similarly designed phantom device. Furthermore, the previously acquired set of digital phantom image data may have been previously acquired using the exact same or similarly designed imaging system. As a result, a “standard” processed set of image data representing a desired level of image quality may be stored in the image data memory of an imaging system and later accessed for comparison with a current set of image processed data using the method 500. In this manner, an imaging system or a plurality of imaging systems may be automatically calibrated to the same standard.

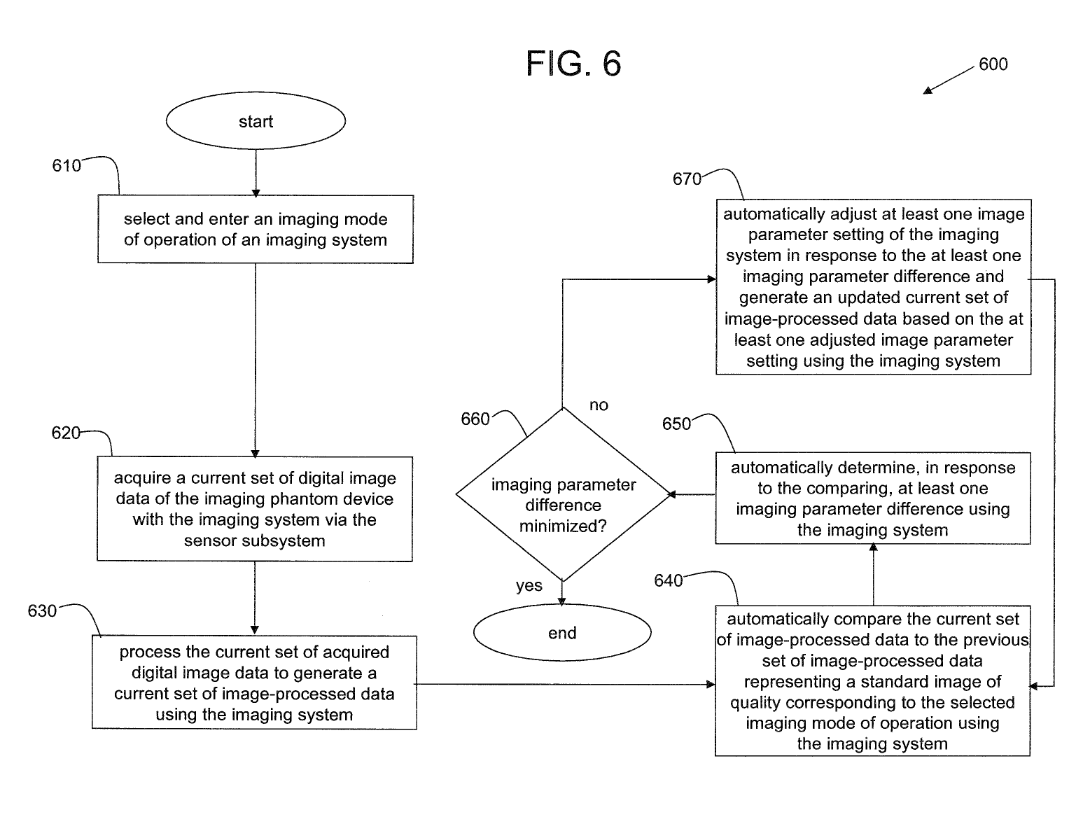

FIG. 6 illustrates a flowchart of a second exemplary embodiment of a method 600 for the automatic adjustment of image parameter settings in the imaging system 300 or 400 of FIG. 3 or FIG. 4 using the imaging phantom device 100 of FIG. 1. The method 600 constitutes an image adjustment or image calibration routine (e.g., the algorithm 459) that may be initiated by an operator or technician of the imaging system. In step 610, select and enter an imaging mode of operation of an imaging system. For example, the imaging modality may be that of diagnostic ultrasound and the selected imaging mode of operation may be that of renal ultrasound. In step 620, acquire a current set of digital image data of the imaging phantom device with the imaging system via the sensor subsystem. In step 630, process the current set of acquired digital image data to generate a current set of image-processed data using the imaging system. In step 640, automatically compare the current set of image-processed data to the previous set of image-processed data representing a standard image of quality corresponding to the selected imaging mode of operation using the imaging system.

In step 650, automatically determine, in response to the comparing, at least one imaging parameter difference using the imaging system. For example, the imaging parameter difference may correspond to a difference in noise between the current set of image-processed data and the previous set of image-processed data (i.e., the standard). In step 660, determine if the at least one imaging parameter difference is minimized (e.g., is below a predetermined minimum threshold value). If the imaging parameter difference is minimized, then end the method 600. If the imaging parameter difference is not minimized, then in step 670, automatically adjust at least one image parameter setting of the imaging system in response to the at least one imaging parameter difference and generate an updated current set of image-processed data based on the at least one adjusted image parameter setting using the imaging system. For example, if the imaging parameter difference corresponds to a difference in noise which is too large (i.e., not minimized), then a noise filter setting may be adjusted. Then go back and repeat steps 640 to 670 until the at least one imaging parameter difference is minimized.

The steps 550 and 640 of automatically comparing may include various sub-steps including spatially aligning the current set of image-processed data with the previously set of image processed data, and performing automatic measurements of various imaging parameters (e.g., noise, brightness, contrast, contrast resolution, spatial resolution, dynamic range, blur, artifacts, and distortion) for both sets of image-processed data using various image processing techniques. Once the various imaging parameters have been determined, imaging parameter differences may be calculated.

If an imaging parameter difference is within a specified acceptable range (e.g., below a pre-defined threshold), then no further action may be necessary. However, if an imaging parameter difference is outside of a specified acceptable range, then the imaging parameter difference is automatically correlated to an image parameter setting which is adjusted based on the imaging parameter difference. In accordance with an embodiment of the present invention, the magnitude of the imaging parameter difference and the associated imaging parameter itself determine which image parameter settings the imaging parameter difference gets correlated to. Furthermore, two or more imaging parameter differences detected during the same test may each get correlated to one or more image parameter settings.

In accordance with an embodiment of the present invention, some examples of correlations may include:
correlating a difference in noise to a noise filter setting;
correlating a difference in brightness to a brightness contrast setting and/or a brightness leveling setting;
correlating a difference in contrast resolution to a contrast leveling setting and/or a gamma setting;
correlating a difference in contrast to a gamma setting and/or a contrast leveling setting;
correlating a difference in color to a HSL or RGB setting.

Other correlations are possible as well, in accordance with various embodiments of the present invention.

The adjusted imaging parameter setting may be displayed on a display device 360 or 460 to an operator or technician performing the calibration. The operator or technician may view the previous set of image-processed data and the updated current set of image-processed data on a display device 360 or 460 and manually confirm that the two displayed images appear acceptably similar to each other.

In accordance with an alternative embodiment of the present invention, instead of comparing the current set of image-processed data to a previous set of image-processed data corresponding to a previously acquired set of digital image data of an imaging phantom device, the current set of image-processed data may be compared to a target set of specifications. The current set of image-processed data is still derived from a current set of digital image data of an imaging phantom device with the imaging system via a sensor subsystem. However, the target set of specifications may simply be a set of heuristics or rules that are applied to the current set of image-processed data to determine how well or how closely the current set of image-processed data corresponds to the rules.

For example, a target set of specifications may specify that a top left portion of the current set of image-processed data should be substantially “white” (e.g., have a gray scale value of between 250 and 255) to be acceptable, a bottom right portion of the current set of image-processed data should be substantially “black” (e.g., have a gray scale value of between 0 to 5) to be acceptable, and a center portion of the current set of image-processed data should be substantially some other consistent color or shade of gray (e.g., have a gray scale value of between 125 and 130) to be acceptable.

The target set of specifications may be stored in an image memory (e.g., 355 or 455) or some other memory of the imaging system and accessed during execution of the algorithm (e.g., 359 or 459) of the imaging system in order to perform a comparison, determine at least one imaging parameter difference, and automatically adjust at least one image parameter setting, if the imaging parameter difference(s) is not minimized (i.e., the specification(s) is not met).

In summary, a system, method, and computer readable medium for facilitating the automatic adjustment of image parameter settings in an imaging system having a sensor subsystem is disclosed. An imaging mode of operation of the imaging system is selected and entered. A current set of digital image data of an imaging phantom device is acquired with the imaging system via the sensor subsystem and processed to generate a current set of image-processed data using the imaging system. The current set of image-processed data is automatically compared to a previous set of image-processed data, representing a standard image of quality corresponding to the selected imaging mode of operation, and at least one image parameter setting may be automatically adjusted to account for a difference in at least one image parameter between the current set of image-processed data and the previous set of image processed data.

While the claimed subject matter of the present application has been described with reference to certain embodiments, it will be understood by those skilled in the art that various changes may be made and equivalents may be substituted without departing from the scope of the claimed subject matter. In addition, many modifications may be made to adapt a particular situation or material to the teachings of the claimed subject matter without departing from its scope. Therefore, it is intended that the claimed subject matter not be limited to the particular embodiment disclosed, but that the claimed subject matter will include all embodiments falling within the scope of the appended claims.

What is claimed is:

1. A method for the automatic adjustment of image parameter settings in an imaging system having a sensor subsystem, providing at least one imaging mode of operation, and storing at least one previous set of image-processed data corresponding to at least one previously acquired set of digital image data of an imaging phantom device and representing a standard image of quality corresponding to said at least one imaging mode of operation, said method comprising:
(a) selecting and entering an imaging mode of operation of said imaging system;
(b) positioning an imaging phantom device with respect to said sensor subsystem;
(c) acquiring a current set of digital image data of said imaging phantom device with said imaging system via said sensor subsystem;
(d) processing said current set of acquired digital image data to generate a current set of image-processed data using said imaging system;
(e) automatically comparing said current set of image-processed data to said previous set of image-processed data representing a standard image of quality corresponding to said selected imaging mode of operation using said imaging system;
(f) automatically determining, in response to said comparing, at least one imaging parameter difference using said imaging system;
(g) automatically adjusting at least one image parameter setting of said imaging system in response to said at least one imaging parameter difference and generating an updated current set of image-processed data based on said at least one adjusted image parameter setting using said imaging system if said at least one imaging parameter difference is not minimized; and
(h) automatically repeating steps (e) through (g) until said at least one imaging parameter difference is minimized.

2. The method of claim 1 wherein said at least one image parameter setting is a noise filter setting of said imaging system.

3. The method of claim 1 wherein said at least one image parameter setting is a brightness contrast setting of said imaging system.

4. The method of claim 1 wherein said at least one image parameter setting is a gamma setting of said imaging system.

5. The method of claim 1 wherein said at least one image parameter setting is a brightness leveling setting of said imaging system.

6. The method of claim 1 wherein said at least one image parameter setting is a contrast leveling setting of said imaging system.

7. The method of claim 1 wherein said at least one image parameter setting is a color setting of said imaging system.

8. A computer readable medium having encoded thereon computer executable instructions for performing a method for the automatic adjustment of image parameter settings in an imaging system having a sensor subsystem, providing at least one imaging mode of operation, and storing at least one previous set of image-processed data corresponding to at least one previously acquired set of digital image data of an imaging phantom device and representing a standard image of quality corresponding to said at least one imaging mode of operation, wherein said method comprises:
(a) selecting and entering an imaging mode of operation of said imaging system;
(b) acquiring a current set of digital image data of said imaging phantom device with said imaging system via said sensor subsystem;
(c) processing said current set of acquired digital image data to generate a current set of image-processed data using said imaging system;
(d) automatically comparing said current set of image-processed data to said previous set of image-processed data representing a standard image of quality corresponding to said selected imaging mode of operation using said imaging system;
(e) automatically determining, in response to said comparing, at least one imaging parameter difference using said imaging system;
(f) automatically adjusting at least one image parameter setting of said imaging system in response to said at least one imaging parameter difference and generating an updated current set of image-processed data based on said at least one adjusted image parameter setting using said imaging system if said at least one imaging parameter difference is not minimized; and
(g) automatically repeating steps (d) through (f) until said at least one imaging parameter difference is minimized.

9. The computer readable medium of claim 8 wherein said at least one image parameter setting is a noise filter setting of said imaging system.

10. The computer readable medium of claim 8 wherein said at least one image parameter setting is a brightness contrast setting of said imaging system.

11. The computer readable medium of claim 8 wherein said at least one image parameter setting is a gamma setting of said imaging system.

12. The computer readable medium of claim 8 wherein said at least one image parameter setting is a brightness leveling setting of said imaging system.

13. The computer readable medium of claim 8 wherein said at least one image parameter setting is a contrast leveling setting of said imaging system.

14. The method of claim 8 wherein said at least one image parameter setting is a color setting of said imaging system.

15. An imaging system having a sensor subsystem, an image processor, and a controller, providing at least one imaging mode of operation, and storing at least one previous set of image-processed data corresponding to at least one previously acquired set of digital image data of an imaging phantom device and representing a standard image of quality corresponding to said at least one imaging mode of operation, and having encoded therein computer executable instructions for performing a method for the automatic adjustment of image parameter settings, wherein said method comprises:
(a) selecting and entering an imaging mode of operation of said imaging system;
(b) acquiring a current set of digital image data of an imaging phantom device with said imaging system via said sensor subsystem;
(c) processing said current set of acquired digital image data to generate a current set of image-processed data;
(d) automatically comparing said current set of image-processed data to said previous set of image-processed data representing a standard image of quality corresponding to said selected imaging mode of operation;
(e) automatically determining, in response to said comparing, at least one imaging parameter difference;
(f) automatically adjusting at least one image parameter setting of said imaging system in response to said at least one imaging parameter difference and generating an updated current set of image-processed data based on said at least one adjusted image parameter setting if said at least one imaging parameter difference is not minimized; and
(g) automatically repeating steps (d) through (f) until said at least one imaging parameter difference is minimized.

16. The imaging system of claim 15 wherein said sensor subsystem includes an X-ray tube with associated transmitting circuitry and an X-ray detector with associated receiving circuitry.

17. The imaging system of claim 15 wherein said at least one image parameter setting is a noise filter setting of said imaging system.

18. The imaging system of claim 15 wherein said at least one image parameter setting is a brightness contrast setting of said imaging system.

19. The imaging system of claim 15 wherein said at least one image parameter setting is a gamma setting of said imaging system.

20. The imaging system of claim 15 wherein said at least one image parameter setting is a brightness leveling setting of said imaging system.

21. The imaging system of claim 15 wherein said at least one image parameter setting is a contrast leveling setting of said imaging system.

22. The method of claim 15 wherein said at least one image parameter setting is a color setting of said imaging system.

23. A method for the automatic adjustment of image parameter settings in an imaging system having a sensor subsystem, providing at least one imaging mode of operation, and storing at least one target specification representing a standard of image quality and corresponding to said at least one imaging mode of operation, said method comprising:
(a) selecting and entering an imaging mode of operation of said imaging system;
(b) positioning an imaging phantom device with respect to said sensor subsystem;
(c) acquiring a current set of digital image data of said imaging phantom device with said imaging system via said sensor subsystem;
(d) processing said current set of acquired digital image data to generate a current set of image-processed data using said imaging system;
(e) automatically comparing said current set of image-processed data to said at least one target specification representing a standard of image quality and corresponding to said selected imaging mode of operation using said imaging system;
(f) automatically determining, in response to said comparing, at least one imaging parameter difference using said imaging system;
(g) automatically adjusting at least one image parameter setting of said imaging system in response to said at least one imaging parameter difference and generating an updated current set of image-processed data based on said at least one adjusted image parameter setting using said imaging system if said at least one imaging parameter difference is not minimized; and
(h) automatically repeating steps (e) through (g) until said at least one imaging parameter difference is minimized.

24. A computer readable medium having encoded thereon computer executable instructions for performing a method for the automatic adjustment of image parameter settings in an imaging system having a sensor subsystem, providing at least one imaging mode of operation, and storing at least one target specification representing a standard of image quality corresponding to said at least one imaging mode of operation, wherein said method comprises:
(a) selecting and entering an imaging mode of operation of said imaging system;
(b) acquiring a current set of digital image data of said imaging phantom device with said imaging system via said sensor subsystem;
(c) processing said current set of acquired digital image data to generate a current set of image-processed data using said imaging system;
(d) automatically comparing said current set of image-processed data to said at least one target specification representing a standard of image quality corresponding to said selected imaging mode of operation using said imaging system;
(e) automatically determining, in response to said comparing, at least one imaging parameter difference using said imaging system;
(f) automatically adjusting at least one image parameter setting of said imaging system in response to said at least one imaging parameter difference and generating an updated current set of image-processed data based on said at least one adjusted image parameter setting using said imaging system if said at least one imaging parameter difference is not minimized; and
(g) automatically repeating steps (d) through (f) until said at least one imaging parameter difference is minimized.

25. An imaging system having a sensor subsystem, an image processor, and a controller, providing at least one imaging mode of operation, and storing at least one target specification representing a standard of image quality corresponding to said at least one imaging mode of operation, and having encoded therein computer executable instructions for performing a method for the automatic adjustment of image parameter settings, wherein said method comprises:
(a) selecting and entering an imaging mode of operation of said imaging system;
(b) acquiring a current set of digital image data of an imaging phantom device with said imaging system via said sensor subsystem;
(c) processing said current set of acquired digital image data to generate a current set of image-processed data;
(d) automatically comparing said current set of image-processed data to said at least one target specification representing a standard of image quality corresponding to said selected imaging mode of operation;
(e) automatically determining, in response to said comparing, at least one imaging parameter difference;
(f) automatically adjusting at least one image parameter setting of said imaging system in response to said at least one imaging parameter difference and generating an updated current set of image-processed data based on said at least one adjusted image parameter setting if said at least one imaging parameter difference is not minimized; and
(g) automatically repeating steps (d) through (f) until said at least one imaging parameter difference is minimized.
